(19) United States
(12) Patent Application Publication
(10) Pub. No.: US 2024/0350049 A1
(43) Pub. Date: Oct. 24, 2024

(54) BLOOD COLLECTION SYSTEM

(71) Applicant: BD KIESTRA B.V., Drachten (NL)

(72) Inventors: Daniel James Robertson, Drachten (NL); Rick van Balen, Drachten (NL)

(73) Assignee: BD KIESTRA B.V., Drachten (NL)

(21) Appl. No.: 18/685,557

(22) PCT Filed: Sep. 6, 2022
(86) PCT No.: PCT/EP2022/074672

§ 371 Date: Feb. 22, 2024

Related U.S. Application Data
(60) Provisional application No. 63/241,352, filed on Sep. 7, 2021.

(51) Int. Cl. A61B 5/15

(52) U.S. Cl. CPC A61B 5/150946; A61B 5/15003; A61B 5/150221

(57) ABSTRACT

A blood metering device for determining an accurate target fill volume of blood in a collection vessel is disclosed herein. The blood metering device includes a housing having an inlet and an outlet, a blood flow conduit defined in the housing for providing a continuous conduit from the inlet to the outlet, and a valve disposed in the blood flow conduit. A valve operation is controlled by a valve actuator for moving the valve from the open position to the closed position. The valve actuator is responsive to a measured gas pressure in a collection vessel in fluid communication with the outlet of the housing where the valve is moved to the closed position when the measured gas pressure is approximately equal to a target gas pressure. A method for determining an accurate target fill volume of blood in a collection vessel is also disclosed herein.

### CROSS-REFERENCE TO RELATED APPLICATIONS

This application claims priority to and the benefit of U.S. Provisional Application No. 63/241,352, which was filed on Sep. 7, 2021, and is incorporated by reference in its entirety herein.

### TECHNICAL FIELD

The present invention relates to blood collection system, and more particularly, to blood collection systems configured to draw blood from a patient and to fill a culture bottle with an accurate predetermined amount of blood.

### BACKGROUND

During blood collection for blood cultures from patients in hospital or other settings, it is important to provide the blood culture bottles with a targeted amount of blood to ensure that the drawn volume is neither too large nor too small, since inoculating the blood culture with an undersized or oversized sample can adversely affect the accuracy of the results of the blood culture analysis. At this moment the only feedback to the medical personnel (typically) drawing blood from a patient is visually monitoring the fluid level in the blood culture bottle during blood draw and discontinuing collection when the fill volume is determined to have been reached.

Currently, the medical personnel make this determination visually. The blood culture bottle has a scale of volume measures on the bottle or the bottle label. Often, the medical personnel are required to mark the target filling volume for the blood on the side of the bottle. In practice, this method is susceptible to error. When a medical professional is drawing blood into the blood culture bottle, the medical personnel may not hold the bottle in a precisely vertical orientation, making it difficult or even impossible to determine the actual volume of the blood collected and making it likely that the target volume of the blood is not obtained. Another issue that can affect the accuracy of the volume of blood drawn is the lack of uniform instructions for how to properly inoculate the blood culture bottle with the target amount of blood. Also, the needs of the patient (who may have difficulties during the blood draw that might distract the medical personnel from accurately monitoring the blood draw) might adversely affect the accuracy of the volume of blood drawn by the medical personnel.

Successfully culturing and detecting a bacteria that has infected a patient is highly dependent on collecting the bacteria in the blood sample taken from the patient. The probability of having bacteria in the blood sample increases with an increase in the volume of blood collected. Therefore, collecting the target volume called for in a blood culture bottle, one example of which is a BACTEC™ culture bottle, with precision, is very important.

As noted above, currently, the medical personnel collecting the blood sample must visually determine when the correct volume of blood has been drawn and collected in the culture bottle, and stop the collection precisely at that point to avoid over-filling the blood culture bottle. Therefore, methods and apparatus for collecting blood that can ensure a target volume of blood is accurately collected continue to be sought.

### BRIEF SUMMARY

Described herein is a blood metering device for determining an accurate target fill volume of blood in a collection vessel. The blood metering device includes a housing having an inlet and an outlet, a blood flow conduit defined in the housing for providing a continuous conduit from the inlet to the outlet, and a valve disposed in the blood flow conduit. A valve operation is controlled by a valve actuator for moving the valve from the open position to the closed position. The valve actuator is responsive to a measured gas pressure in a collection vessel in fluid communication with the outlet of the housing where the valve is moved to the closed position when the measured gas pressure is approximately equal to a target gas pressure.

Also described herein is a method for determining an accurate target fill volume of blood in a collection vessel with the blood metering device described above. The method includes connecting the blood metering device to the collection vessel such that the blood metering device is in fluid communication with the collection vessel, inputting the target fill volume to the blood metering device, determining a target gas pressure in the collection vessel, collecting a blood sample from a patient by venipuncture, thereby causing blood to flow through the blood metering device to the collection vessel, repeatedly measuring a gas pressure in the collection vessel as blood flows into the collection vessel, repeatedly comparing the measured gas pressure with the target gas pressure, and stopping the blood from flowing into the collection vessel when the measured gas pressure is equal to the target gas pressure.

These and other aspects of the present invention will be better understood in view of the drawings and following detailed description.

### BRIEF DESCRIPTION OF DRAWINGS

FIG. 1 is a perspective view of a blood collection system, according to an embodiment of the present invention.

FIG. 2 is a perspective view of the blood metering device of the blood collection system of FIG. 1.

FIG. 3 is a transparent perspective view of the blood metering device of the blood collection system of FIG. 1.

FIG. 4 is an exploded perspective view of the blood metering device of the blood collection system of FIG. 1.

FIG. 5 is a perspective view of a bottom portion of the blood metering device of the blood collection system of FIG. 1 with the valve in a closed position.

FIG. 6 is a perspective view of a bottom portion of the blood metering device of the blood collection system of FIG. 1 with the valve in an open position.

FIG. 7 is a side view of the valve disposed within the blood flow conduit of the blood metering device in an open position.

FIG. 8 is a blood flow path through the blood metering device of the blood collection system of FIG. 1.

FIG. 9 is a flow chart of a method for determining an accurate blood fill volume in a blood collection vessel, according to an embodiment of the present invention.

### DETAILED DESCRIPTION

Embodiments of the present disclosure are described in detail with reference to the drawing figures wherein like reference numerals identify similar or identical elements. It is to be understood that the disclosed embodiments are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

The blood metering device described herein collects blood from a patient and fills a blood collection vessel or collection bottle, which the device is attached, with an accurate volume of blood. The collection bottle is any suitable container for receiving a blood sample. One example is a blood collection tube such as a BD Vacutainer® tube. BD Vacutainer is a registered trademark of Becton, Dickinson and Company. Another example is a blood culture bottle such as the BACTEC bottle described above. The blood metering device provides at least one of: 1) an indication when a target volume of blood has passed through the device and into the collection bottle; or 2) an automatic shut off when a target volume of blood has passed through the device and into the collection bottle.

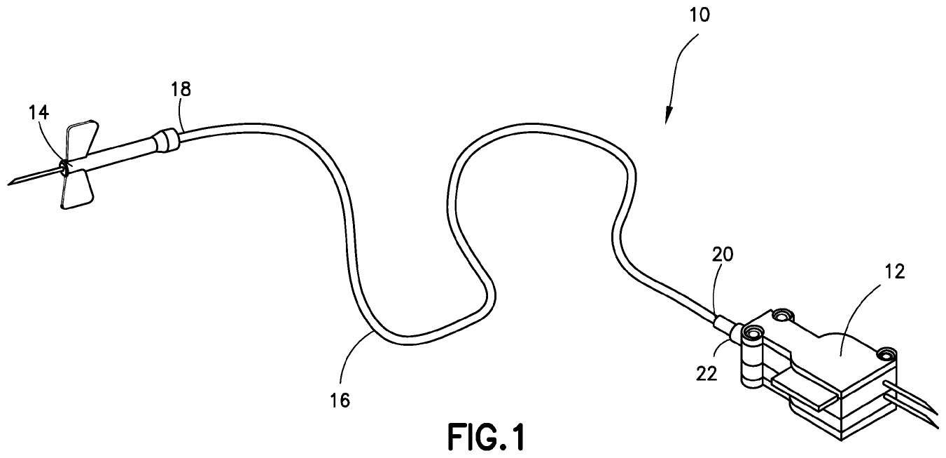

FIG. 1 illustrates a blood collection system 10 comprising a blood metering device 12 in accordance with the present technology. The blood collection system 10 includes a butterfly needle 14, a tubing 16, and a blood metering device 12 connected to the butterfly needle 14 via the tubing 16. Specifically, the butterfly needle 14 is connected to a first end 18 of the tubing 16 and the blood metering device 12 is connected to a second end 20 of the tubing 16 via a tubing connector 22.

Directional terms, such as top, bottom, up and down, are referenced to an orientation in which the blood metering device 12 is connected to a collection vessel (not shown) placed on a flat surface. However, the present invention is not thereby limited to use in any particular orientation.

During the process of collecting a blood sample from a patient, the butterfly needle 14 is used to pierce a vein or an artery of the patient. Driven by the vacuum pressure created by the collection vessel, blood from the patient is directed toward the collection vessel through the tubing 16. A flow of blood is collected in the collection vessel. Along the way, the blood passes through the blood metering device 12.

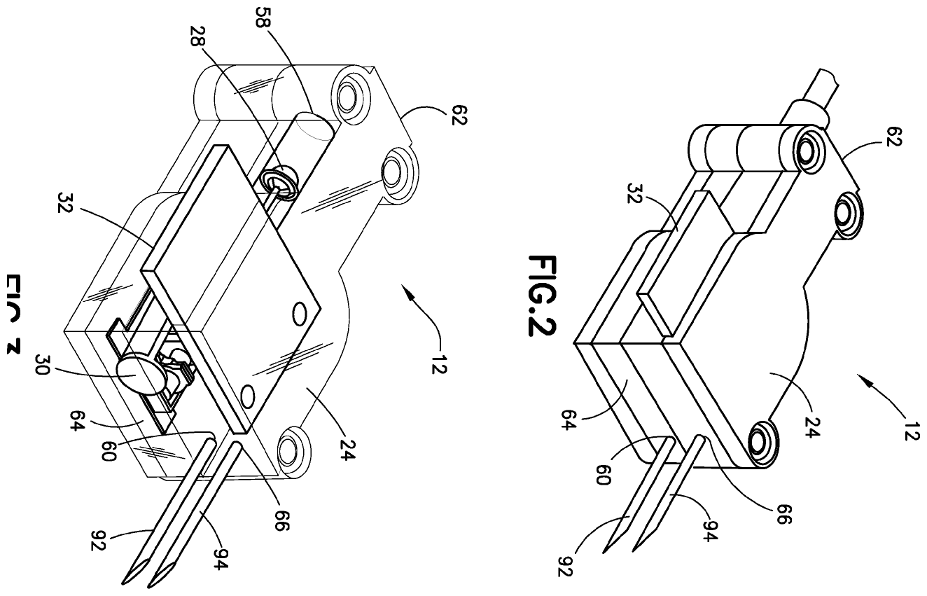

Referring to FIGS. 2-5, the blood metering device 12 includes a housing 24 in which is disposed a blood flow conduit 26 for blood from a patient to flow therethrough, a valve 28 for controlling the blood flow into the collection vessel, a valve actuator 30 for facilitating the valve 28, a printed circuit board (“PCB”) 32 for controlling various components in the blood metering device 12, a pressure sensor (not shown) connected to the PCB 32, and a battery (not shown).

The PCB 32 includes a microcontroller having a processor and a memory therewithin, and other electronics necessary to facilitate various components of the blood metering device 12. For example, the processor can actuate the valve actuator 30 to open the valve 28 to commence the blood collection process and close the valve 28 once the predetermined fill volume (or target fill volume) has been filled in the collection vessel. The memory stores information therein that controls the operation of the blood metering device 12. Non-limiting examples of such information includes total blood volume that passes through the blood metering device (i.e., the predetermined fill volume) 12, the maximum duration of the blood draw (after which time the blood metering device 12 terminates further collection of the blood from the patient), and changes in blood flow rate from the patient indicative of vein collapse. In addition, the microcontroller provides blood collection process information to the user via a LED (not shown) installed on the blood metering device 12. For example, the LED provides an indication of blood volume that has passed through the blood metering device 12 and reached the predetermined fill volume. Other indicators that the predetermined fill volume has been received by the collection vessel include sensory alerts such as a vibration alert.

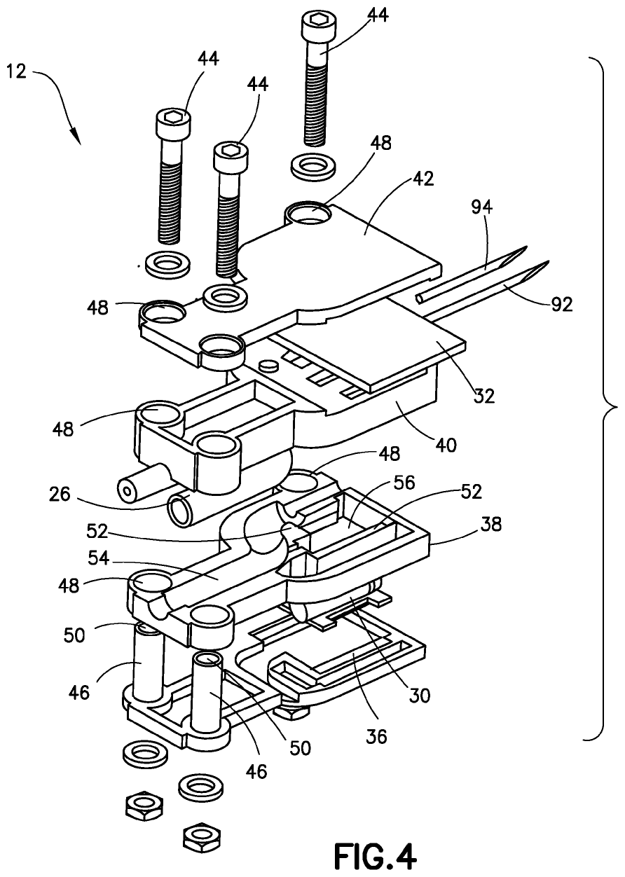

Referring particularly to FIG. 4, the housing 24 includes a lower casing 36, a lower chassis 38, an upper chassis 40, and an upper casing 42. These are the housing components of the blood metering device 12 that are configured to be coupled together via a plurality of fasteners (e.g., nuts and bolts as illustrated) 44 to form an external housing of the blood metering device 12. For example, in the illustrated embodiment, the housing 24 is assembled by stacking up each of the housing components 36, 38, 40, 42. Specifically, the lower chassis 38 is mounted onto the lower casing 36 by aligning and inserting a plurality of connecting poles 46 defined on the lower casing 36 to a plurality of connecting apertures 48 defined on the lower chassis 38. Each of the plurality of connecting poles 46 include a connecting hole 50 extending therethrough for receiving and accommodating the fastener 44 therewithin. The upper chassis 40 and upper casing 42 are placed onto the lower chassis 38 and the upper chassis 40, respectively, in a similar manner. One of ordinary skill will envision other fastening mechanisms such as glue, snap fit, etc. The fastening mechanisms illustrated in the figures are for purposes of illustration only.

Once all of the housing components 36, 38, 40, 42 are assembled, each of the plurality of fasteners 44 are inserted into the connection hole 50 of the respective connecting pole 46 for coupling the housing components and forming an external housing of the blood metering device 12. Although the blood metering device 12 in the illustrated embodiment has four housing components (upper casing, upper chassis, lower chassis, and lower casing), the housing 24 may have other configurations that have a different number of housing components, e.g., two housing components with lower and upper casings 36, 42.

The lower chassis 38 and upper chassis 40 provide a structural skeleton for the blood metering device 12. The lower chassis 38 includes a plurality of dividers (or walls) 52 for defining various compartments to provide discrete sections/flow channels for various inner components of the blood metering device 12. For example, a first compartment 54 is defined within the lower chassis 38 for the blood flow conduit 26 to be seated thereon while a second compartment 56 is configured to receive the valve actuator 30 therewithin.

Contours of the top section of the lower chassis 38 and the bottom section of the upper chassis 40 are configured and dimensioned such that, when engaged and assembled, the first and second compartments 54, 56 are fully formed for receiving the blood flow conduit 26 and the valve actuator 30, respectively. In addition, contours of the top section of the upper chassis 40 and the bottom section of the upper casing 42 are configured and dimensioned such that, when engaged and assembled, the PCB 32 and pressure sensor may be disposed and secured therebetween.

Once the housing components 36, 38, 40, 42 are fully assembled, an inlet 58 of the blood metering device 12 and an outlet 60 of the blood metering device 12 are formed at first and second ends 62, 64 of the blood metering device 12, respectively, as shown in FIGS. 2 and 3. The inlet 58 allows the blood drawn from the patient to enter the blood metering device 12 whereas the outlet 60 allows the blood to exit the blood metering device 12. In addition, a pressure sensor hole 66 is formed at the second end of the blood metering device 12.

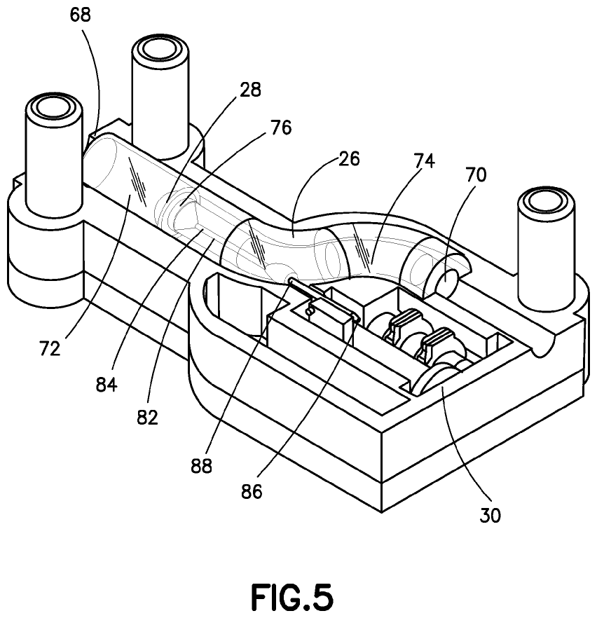

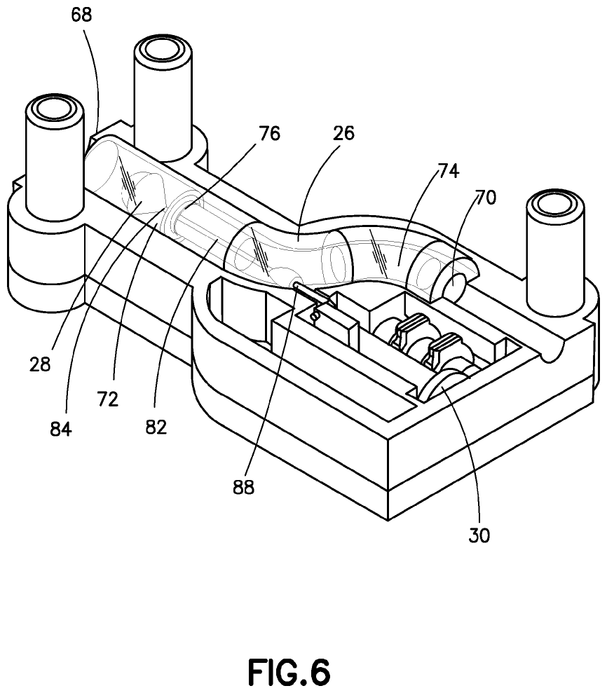

Referring to FIGS. 5 and 6, the blood flow conduit 26 extends between a first open end 68 and a second open end 70 thereof. The blood flow conduit 26 is connected to and is in fluid communication with the tubing 16, via the tubing connector 22, at the inlet 58 of the blood metering device 12. The blood flow conduit 26 defines a passageway therewithin for the drawn blood to travel therethrough and to the collection vessel. The passageway of the blood flow conduit 26 extends between the first open end 68 of the blood flow conduit (or at the inlet 58 of the blood metering device 12) 26 and the second open end 70 of the blood flow conduit 26.

The blood flow conduit 26 includes a first portion 72, a second portion 74, and a transition portion 76 connecting the first and second portions 72, 74. The first portion 72 of the blood flow conduit 26 has a first inner diameter that is larger than a second inner diameter of the second portion 74 such that the passageway of the first portion 72 is wider than the passageway of the second portion 74.

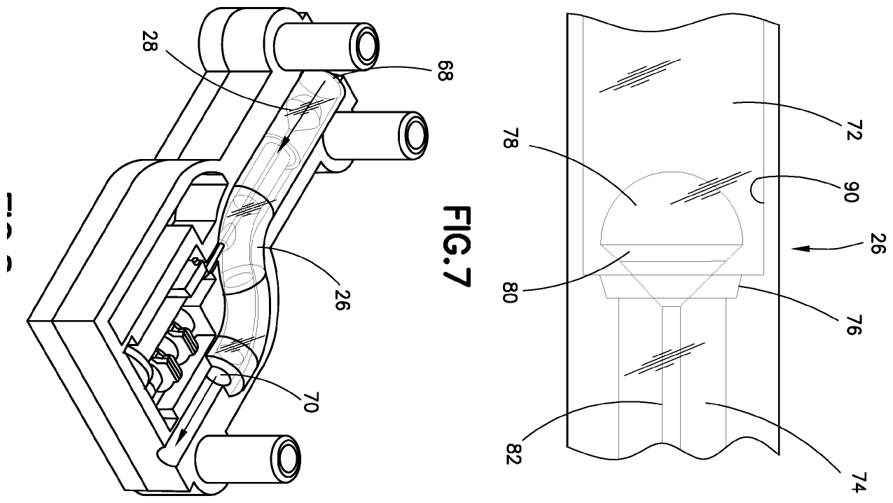

Referring to FIG. 7, the transition portion 76 is frustrum-shaped and connects the first portion 72 at one end and the second portion 74 at the other end. This configuration, with contours of the valve 28, allows the valve 28 to engage with the transition portion 76 and to transition between an open position to allow blood to flow through the second portion 74 of the blood flow conduit 26 and a closed position to stop the blood flow from flowing into the second portion 74 of the blood flow conduit 26, as will be described in greater detail below.

Referring back to FIGS. 5-7, the valve 28 is disposed within the first portion 72 of the blood flow conduit 26 and includes a sealing portion and a rod portion. The sealing portion has a dome shaped top section 78 and a conical shaped bottom section 80. As stated above, the valve 28 transitions between the open and closed positions to control the blood flow. The valve 28 is connected to the valve actuator 30 via the rod portion 82. The rod portion 82 extends between a first end 84 and a second end 86 thereof and through a hole 88 defined on the second portion 74 of the blood flow conduit 26. The rod portion 82 connects to the valve 28 at the first end 84 and connects with the valve actuator 30 at the second end 86. Thus, the valve actuator 30 facilitates axial movements of the valve 28 within the first portion 72 of the blood flow conduit 26 and transitions the valve 28 between the open and closed positions.

A diameter of the top section 78 of the valve 28 is less than the first inner diameter of the first portion 72 of the blood flow conduit 26 such that a clearance exists between the top section 78 of the valve 28 and an inner surface 90 of the blood flow conduit 26 when the valve 28 is in the open position, as shown in FIG. 7. The clearance allows the blood to continue to flow from the first portion 72 of the blood flow conduit 26 to the second portion 74 of the blood flow conduit 26 and to the collection vessel.

In the illustrated embodiment, the valve actuator 30 controls the flow of blood collected from the patient by keeping the valve 28 closed when blood draw from the patient commences. After blood draw is commenced, the valve actuator 30 receives a signal from the microcontroller indicating that blood flow has started. In response to such signal, the valve actuator 30 gradually causes the valve 28 to open. Specifically, the valve actuator 30 moves the valve 28 axially upward and thereby causing the sealing portion of the valve 28 to disengage with the transition portion 76 of the blood flow conduit 26. Thus, the blood can flow around the valve 28, through the clearance and to the second portion 74 of the blood flow conduit 26. In addition, the valve actuator 30 is programmed, via the microcontroller, to open the valve 28 in a manner that mitigates hemolysis of the blood flowing through the blood flow conduit 26.

Once the predetermined fill volume of blood has been filled in the collection vessel, the microcontroller again sends a signal to the valve actuator 30 indicating that the predetermined fill volume has been reached. In response to such signal, the valve actuator 30 causes the valve 28 to close and automatically shut the blood metering device 12 off. Specifically, the valve actuator 30 moves the valve 28 axially downward and thereby causing the sealing portion of the valve 28 to engage with the transition portion 76 of the blood flow conduit 26. An upper section of the conical shaped bottom section 80 of the valve 28 is fully disposed within the transition portion 76 to close the opening to the second portion 74 of the blood flow conduit 26.

Suitable valve actuators are well known to one skilled in the art and are not described in detail herein. Such actuators include moving magnet actuators, micro actuators, solenoids, paired magnets, etc. that, in response to a signal, cause the valve to open or close.

Other suitable valves for the blood metering device include a shut off valve, the pinch tube valve, ball valves, membrane valves, slide valves, check valves, release valves, etc.

In an alternate embodiment, the blood metering device 12 is configured to operate without the valve and valve actuator. As stated above, the microcontroller provides blood collection process information to the user via an indicator (e.g., LED, vibration alert, etc.) installed on the blood metering device 12. For example, the indicator provides an indication of blood volume that has passed through the blood metering device 12 and reached the predetermined fill volume (or target fill volume). Once the blood metering device 12 indicates that the predetermined fill volume has reached, the user may stop the blood from flowing into the blood collection vessel by manually shutting off the blood metering device 12.

Referring back to FIGS. 2-4, the blood metering device 12 further includes a first needle 92 for filling blood in the collection vessel and a second needle 94 for measuring pressure of the gas in the collection vessel. The first needle 92 of the blood metering device 12 is connected to the second open end 70 of the blood flow conduit 26 and extends through the outlet 60 of the blood metering device 12 for blood to flow into and fill the collection vessel.

The second needle 94 is connected to the pressure sensor and extends through the pressure sensor hole 66 of the blood metering device 12 for measuring pressure of the gas in the collection vessel. The first and second needles 92, 94 may pierce through a cap of the collection vessel (not shown) to connect the blood metering device 12 to the collection vessel.

Alternatively, the blood metering device 12 may include a double lumen needle for filling blood in the collection vessel and measuring pressure of the gas in the collection vessel.

The blood metering device 12 is made of one or more materials having suitable properties for a desired application, including strength, weight, rigidity, etc. Plastic (e.g., polypropylene, polyethylene, etc.) is preferred for the housing 24 of the blood metering device 12, and a silicone tube is preferred for the blood flow conduit 26.

The blood flow path through the blood metering device 12 is illustrated in FIGS. 3 and 8. The blood enters the blood metering device 12 through the inlet 58 via the tubing 16. The blood flow path enters the passageway of the blood flow conduit 26 from the first open end 68 of the blood flow conduit 26 and travels therethrough when the valve 28 is open, and exits the passageway from the second open end 70 of the blood flow conduit 26. After exiting the blood flow conduit 26, the blood continues to flow to the collection via the first needle 92.

As stated above, the blood metering device 12 accordance with the present technology is configured to measure fill level of the collection vessel (e.g., volume of the blood in the collection vessel) by measuring the gas pressure in the collection vessel using the pressure sensor. Specifically, as the blood fills the collection vessel, the gas pressure in the collection vessel decreases, which is caused by addition of the blood to the collection vessel. Thus, the blood collection system 10 measures the degree of vacuum before and during the blood collection process to estimate the amount of blood added to the collection vessel at a specific time. This estimation of the amount of blood added to the collection vessel may be calculated using Boyle's law, which states that the pressure of a given quantity of gas varies inversely with its volume. The estimation may be determined using the Boyle's law since the total volume of the collection vessel and the total amount of gas in the collection vessel do not change.

The Boyle's law equation states:

$${P}_{0} * {V}_{0} = {P}_{1} * {V}_{1} = K \qquad ( 1 )$$

where P₀ is initial pressure, P₁ is pressure at time t₁, V₀ is initial volume of gas, V₁ is volume of gas at t₁, and K is a constant. In the claimed invention, P₀ is pressure of the gas in the collection vessel at start time of blood fill (t₀) (or pressure in the collection vessel before collecting blood sample from a patient), P₁ is pressure of the gas in the collection vessel at end time of blood fill (t₁), V₀ is volume of gas in the headspace of the collection vessel at t₀ (or gas volume in the collection vessel before collecting blood sample from a patient), and V₁ is volume of gas in the headspace of the collection vessel at t₁. The headspace is equal to the total volume of the collection vessel minus the total volume of the broth disposed within the collection vessel. The constant (K) is then determined by the following equation:

$$K = {V}_{0} * {P}_{0} \qquad ( 2 )$$

The volume of gas at t₁ (V₁) is calculated by:

$${V}_{1} = {V}_{0} - {V}_{D} \qquad ( 3 )$$

where V_{D} is the desired amount of blood (e.g., 8 mL, 10 mL, etc.) to be filled (or predetermined (target) fill volume) within the collection vessel. Now, the pressure at t₁ (P₁) is determined by the following equation:

$${P}_{1} = K / {V}_{1} = {V}_{0} * {P}_{0} / ( {V}_{0} - {V}_{D} ) \qquad ( 4 )$$

Thus, V_{D} is reached in the collection vessel when the gas pressure of the collection vessel is at P₁.

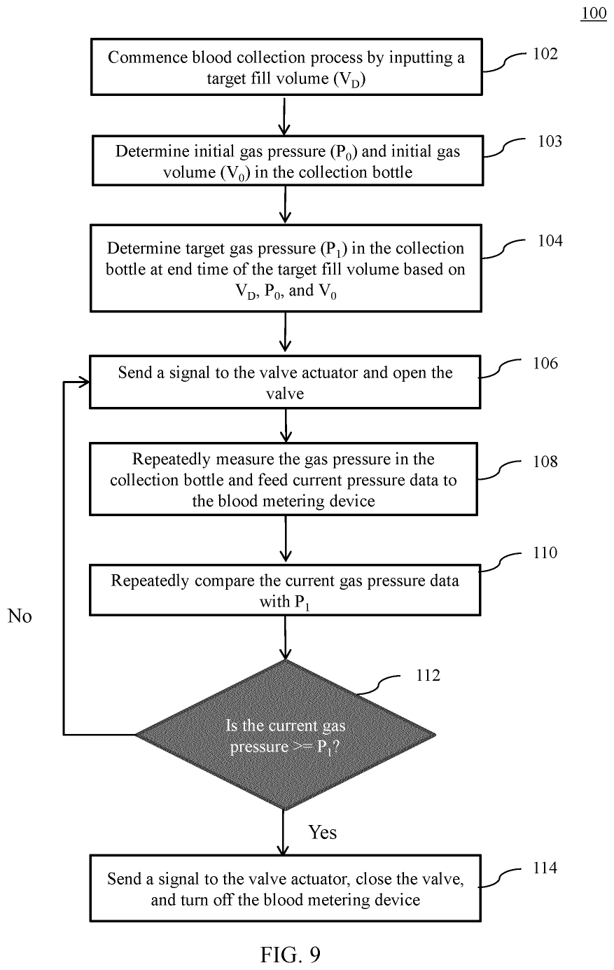

FIG. 9 illustrates a method 100 of determining an accurate blood fill volume in a blood collection vessel using the blood collection system 10 in accordance with the present technology. At step 102, a blood collection process is commenced by inputting (or selecting) a target (or predetermined) fill volume (V_{D}) to the blood metering device 12 by a user. At step 103, the metering device 12 determines the initial gas pressure (P₀) and initial gas volume (V₀) in the collection vessel. By using the above described pressure sensor fill algorithm based on the Boyle's law, at step 104, the microcontroller of the blood metering device 12 determines the target pressure (P₁) of the gas in the collection vessel at the end time of the target fill volume based on V_{D}, P₀, and V₀. At step 106, a signal is sent to the valve actuator 30 to open the valve 28 to permit blood drawn from a patient to flow through the blood metering device 12. At step 108, the pressure sensor repeatedly measures the gas pressure in the collection vessel via the second needle 94 placed in the collection vessel and feeds current pressure data to the microcontroller of the blood metering device 12. At step 110, the blood metering device 12 repeatedly compares the received gas pressure data (e.g., current gas pressure in the collection vessel) with the target gas pressure (P₁). If the blood metering device 12 determines that the current gas pressure in the collection vessel is equal to or greater than P₁ at step 112, a signal is sent to the valve actuator 30 to close the valve 28 to stop the blood flow, and the blood metering device 12 turns off automatically (at step 114). If, however, the blood metering device 12 determines that the current gas pressure in the collection vessel is less than P₁ at step 112, the valve 28 is kept open to permit the blood to keep traveling through the blood metering device 12 and to the collection vessel.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

1. A method for determining an accurate target fill volume of blood in a collection vessel, the method comprising:
providing a blood metering device comprising:
a housing comprising an inlet and an outlet; and
a blood flow conduit disposed in the housing, the blood flow conduit providing a continuous conduit from the inlet to the outlet;
connecting the blood metering device to the collection vessel such that the blood metering device is in fluid communication with the collection vessel;
inputting the target fill volume to the blood metering device;
determining a target gas pressure in the collection vessel;
collecting a blood sample from a patient by venipuncture, thereby causing blood to flow through the blood metering device to the collection vessel;
repeatedly measuring a gas pressure in the collection vessel as blood flows into the collection vessel;
repeatedly comparing the measured gas pressure with the target gas pressure; and
stopping the blood from flowing into the collection vessel when the measured gas pressure is equal to the target gas pressure.

2. The method of claim 1, wherein the target gas pressure indicates that the target fill volume of blood has entered the collection vessel.

3. The method of claim 1, further comprising, before determining a target gas pressure in the collection vessel, determining an initial gas pressure in the collection vessel and initial gas volume in the collection vessel.

4. The method of claim 3, wherein the initial gas pressure is a pressure in the collection vessel before collecting the blood sample from the patient.

5. The method of claim 3, wherein the initial gas volume is a gas volume in the collection vessel before collecting the blood sample from the patient.

6. The method of claim 3, wherein the target gas pressure is determined based on the target fill volume, initial gas pressure, and initial gas volume.

7. The method of claim 1, wherein the target fill volume is a desired amount of blood to be filled within the collection vessel.

8. The method of claim 1, wherein the measured gas pressure decreases as the blood flows into the collection vessel.

9. A blood metering device for determining an accurate target fill volume of blood in a collection vessel, the blood metering device comprising:
a housing comprising an inlet and an outlet;
a blood flow conduit defined in the housing, the blood flow conduit providing a continuous conduit from the inlet to the outlet; and
a valve disposed in the blood flow conduit wherein a valve operation is controlled by a valve actuator for moving the valve from an open position to a closed position, wherein the valve actuator is responsive to a measured gas pressure in a collection vessel in fluid communication with the outlet of the housing where the valve is moved to the closed position when the measured gas pressure is approximately equal to a target gas pressure.

10. The blood metering device of claim 9, wherein the target gas pressure in the collection vessel indicates that the target fill volume of blood has entered the collection vessel.

11. The blood metering device of claim 9, wherein the target gas pressure is determined based on the target fill volume, initial gas pressure, and initial gas volume.

12. The blood metering device of claim 11, wherein the target fill volume, initial gas pressure, and initial gas volume are a desired amount of blood to be filled within the collection vessel, a pressure in the collection vessel before collecting a blood sample from a patient, and a gas volume in the collection vessel before collecting the blood sample from the patient, respectively.

13. The blood metering device of claim 9, wherein the blood flow conduit includes a first portion with a first inner diameter, a second portion with a second inner diameter, and a transition portion from the first inner diameter to the second inner diameter.

14. The blood metering device of claim 13, wherein the first inner diameter is larger than the second inner diameter.

15. The blood metering device of claim 13, wherein the valve comprises a sealing portion and a rod portion operable from the open position to the closed position.

16. The blood metering device of claim 15, wherein the valve blocks flow of blood in the blood flow conduit in the closed position by drawing the sealing portion into the transition portion, thereby sealing the first portion off from the second portion.

17. The blood metering device of claim 13, wherein the transition portion is frustrum-shaped and connects the first portion at one end and the second portion at the other end.

18. The blood metering device of claim 15, wherein the rod portion connects the valve and valve actuator and extends through a hole defined on the second portion of the blood flow conduit.

19. The blood metering device of claim 9, further comprising a first needle for filling blood in the collection vessel and a second needle for measuring gas pressure in the collection vessel.

20. The blood metering device of claim 9, further comprising a double lumen needle for filling blood in the collection vessel and measuring gas pressure in the collection vessel.
